(19) United States
(12) Patent Application Publication
(10) Pub. No.: US 2025/0361243 A1
(43) Pub. Date: Nov. 27, 2025

(54) Compounds and Pharmaceutical Compositions Useful for Managing Sickle Cell Disease and Conditions Related Thereto

(71) Applicants: Emory University, Atlanta, GA (US); Children's Healthcare of Atlanta, Inc., Atlanta, GA (US)

(72) Inventors: Eric J. Sorscher, Atlanta, GA (US); Wook Joon Chung, Decatur, GA (US); Huw M. L. Davies, Suwanee, GA (US); Yuhong Du, Atlanta, GA (US); Yannick Boni, Stone Mountain, GA (US); Haian Fu, Decatur, GA (US); David Archer, Roswell, GA (US)

(21) Appl. No.: 18/867,384

(22) PCT Filed: May 19, 2023
(86) PCT No.: PCT/US2023/022912

§ 371 Date: Nov. 19, 2024

Related U.S. Application Data
(60) Provisional application No. 63/344,233, filed on May 20, 2022.

(51) Int. Cl. C07D 513/04; A61K 31/402; A61K 31/4025; A61K 31/404; A61K 31/437; A61K 31/4439; A61K 31/4709; A61P 7/06; C07D 207/416; C07D 207/448; C07D 209/38; C07D 401/04; C07D 403/04

(52) U.S. Cl. CPC C07D 513/04; A61K 31/402; A61K 31/4025; A61K 31/404; A61K 31/437; A61K 31/4439; A61K 31/4709; A61P 7/06; C07D 207/416; C07D 207/448; C07D 209/38; C07D 401/04; C07D 403/04

(57) ABSTRACT

This disclosure relates to compounds, pharmaceutical compositions, and methods of treating or preventing sickle cell disease or conditions associated thereto. In certain embodiments, the compounds are 1-(thiazol-2-yl)urea derivatives. In certain embodiments, the compounds are 1-(9H-carbazol-9-yl)-3-mercaptopropan-2-ol derivatives. In certain embodiments, the compounds are 1-phenyl-1H-pyrrole-2,5-dione derivatives. In certain embodiments, this disclosure relates to methods of treating or preventing sickle cell disease or condition associated thereto comprising administering an effective amount of a 1-(thiazol-2-yl)urea derivative, or 1-phenyl-1H-pyrrole-2,5-dione derivative, or a 1-(9H-carbazol-9-yl)-3-mercaptopropan-2-ol derivative as disclosed herein to a subject in need thereof.

### CROSS-REFERENCE TO RELATED APPLICATIONS

This application claims the benefit of U.S. Provisional Application No. 63/344,233 filed May 20, 2022. The entirety of this application is hereby incorporated by reference for all purposes.

### BACKGROUND

Hemoglobin is a protein in red blood cells that carries oxygen throughout the body. Sickle cell disease (SCD) is a hereditary red blood cell disorder, which results from a congenital mutation in the beta-globin chain leading to the production of a defective form of hemoglobin often referred to as hemoglobin S (HbS). When a person has two hemoglobin S genes, Hemoglobin SS (Hb SS), the disease is called sickle cell anemia, which is the most common and often most severe kind of SCD.

Sickle hemoglobin differs from normal hemoglobin in its propensity to form polymers under conditions of low oxygen tension, changing the red blood cell into a crescent, or sickle shape. Sickle-shaped cells are not flexible, which can cause a blockage that slows or stops the flow of blood and essentially obstructs the microcirculation. When this happens, oxygen cannot reach nearby tissues. The lack of oxygen can cause attacks of sudden, severe pain, called vaso-occlusive crisis (VOC), pain crisis, or sickle cell crisis, which results in ischemic injury to organs. Pain crises constitute a distinguishing clinical feature of VOC of SCD and are a leading cause of emergency department visits and hospitalizations for affected patients.

Current treatment of sickle cell disease and for clinical complications include the use of fluids, oxygen, analgesics, hydroxyurea, red blood cell (RBC) transfusions, and hematopoietic stem cell transplantation (HSCT). Voxelotor (Oxbryta™) is a clinically approved anti-sickling drug that increases blood hemoglobin levels and diminishes red cell transfusion requirements; however, it has exhibited insufficient activity against several clinical endpoints. Thus, improved treatment strategies are needed.

### SUMMARY

This disclosure relates to compounds, pharmaceutical compositions, and methods of treating or preventing sickle cell disease or conditions associated thereto. In certain embodiments, the compounds are 1-(thiazol-2-yl)urea derivatives. In certain embodiments, the compounds are 1-(9H-carbazol-9-yl)-3-mercaptopropan-2-ol derivatives. In certain embodiments, the compounds are 1-phenyl-1H-pyrrole-2,5-dione derivatives. In certain embodiments, this disclosure relates to methods of treating or preventing sickle cell disease or condition associated thereto comprising administering an effective amount of a 1-(thiazol-2-yl)urea derivative, or 1-phenyl-1H-pyrrole-2,5-dione derivative, or a 1-(9H-carbazol-9-yl)-3-mercaptopropan-2-ol derivative as disclosed herein to a subject in need thereof.

In certain embodiments, this disclosure relates to using a compound disclosed herein for treating subjects with sickle cell disease, beta thalassemia, or variants or related diseases or conditions thereof.

In certain embodiments, this disclosure relates to using a compound disclosed herein for reducing one or more symptoms of sickle cell disease, beta thalassemia, or variants or related diseases or conditions thereof.

In certain embodiments, this disclosure relates to using a compound disclosed herein for treating vaso-occlusive crisis or related complications. In certain embodiments, this disclosure relates to using a compound disclosed herein for treating sickle cell-related retinopathy.

In certain embodiments, the method further comprises co-administering hydroxyurea, crizanlizumab, arginine, voxelotor, etavopivat, mitapivat, and/or combinations or another therapeutic agent to the subject.

In certain embodiments, this disclosure relates to pharmaceutical compositions comprising a compound as disclosed herein or salt thereof and a pharmaceutically acceptable excipient. In certain embodiments, the pharmaceutical composition is in the form of a tablet, capsule, gel, gel capsule, or cream. In certain embodiments, the pharmaceutical composition is in the form of a pH buffered saline solution optionally comprising a saccharide or polysaccharide.

In certain embodiments, this disclosure relates to the production of a medicament for use in treating or preventing sickle cell disease or condition associated thereto.

In certain embodiments, this disclosure relates to methods of making compounds disclosed herein by mixing starting materials with reagents under conditions such that the products are formed.

### BRIEF DESCRIPTION OF THE SEVERAL VIEWS OF THE DRAWINGS

FIG. 1A illustrates the chemical structures and names of examples of this disclosure.

FIG. 1B illustrates the chemical structures and names of examples of this disclosure.

FIG. 1C illustrates the chemical synthesis of the compounds and derivatives from appropriately substituted starting materials.

FIG. 1D illustrates the chemical structure and name of example of this disclosure.

FIG. 2 shows data indicating SCAS2_05 is able to dose dependently prevent sickling of RBCs from sickle cells patients. Treatment (50 μM) can fully restore round shape morphology compared to normoxic conditions and when compared to control drug (5 mM 5-HMF).

FIG. 3 shows data on cytotoxic effects of anti-sickling agents on HEK293 cells. Cultured HEK293 cells in a 96-well plate incubated with various concentrations of anti-sickling agents overnight in a CO2 incubator. After incubation, media from the culture were collected and incubated with reductase substrate (LDH-glo™ Cytotoxicity assay kit, Promega) to generate bioluminescence in the presence of LDH released from damaged cells. Bioluminescence was measured and normalized to a positive control (membrane permeabilized with 0.1% Triton X-100).

FIG. 4 illustrates the chemical structures and names of examples of this disclosure.

FIG. 5A shows data on compounds of this disclosure.

FIG. 5B illustrates the chemical structures and names of examples of this disclosure.

FIG. 5C illustrates the chemical structures and names of examples of this disclosure.

FIG. 5D illustrates the chemical structures and names of examples of this disclosure.

FIG. 5E illustrates a method of making compounds of this disclosure.

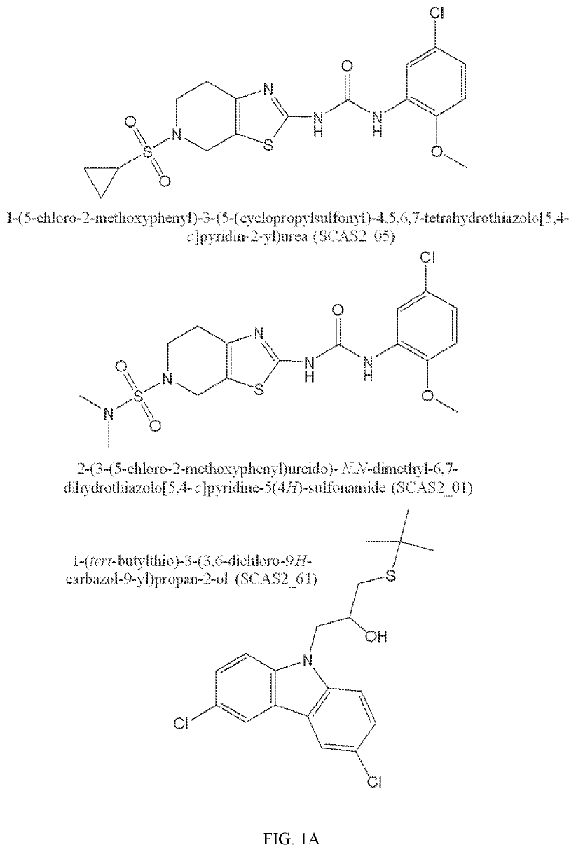

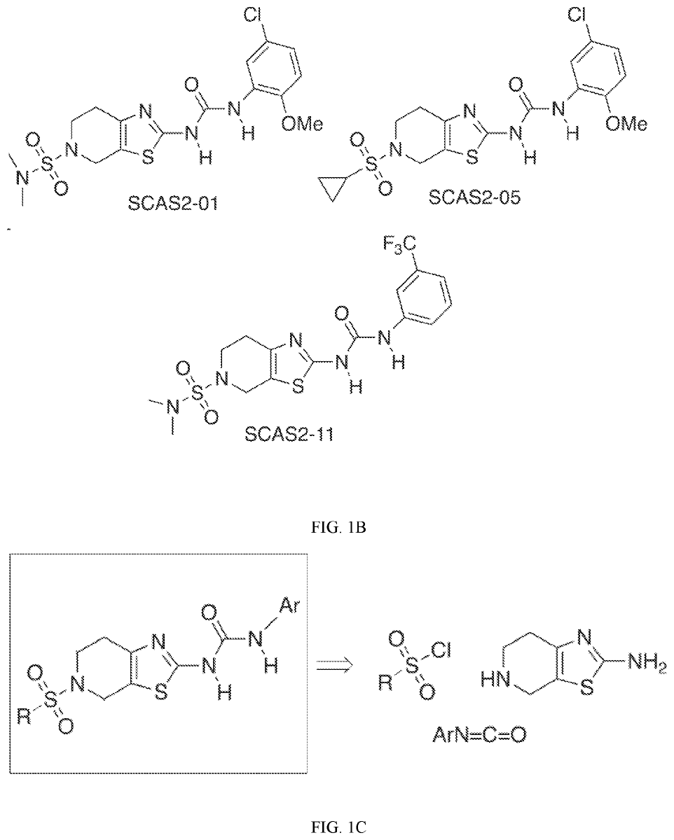

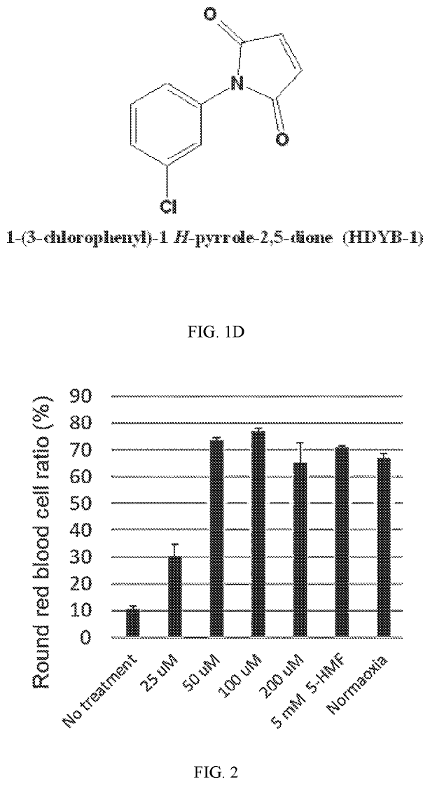

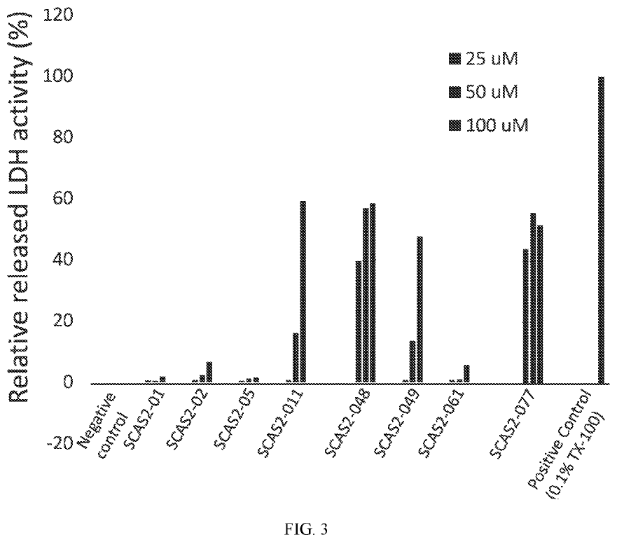

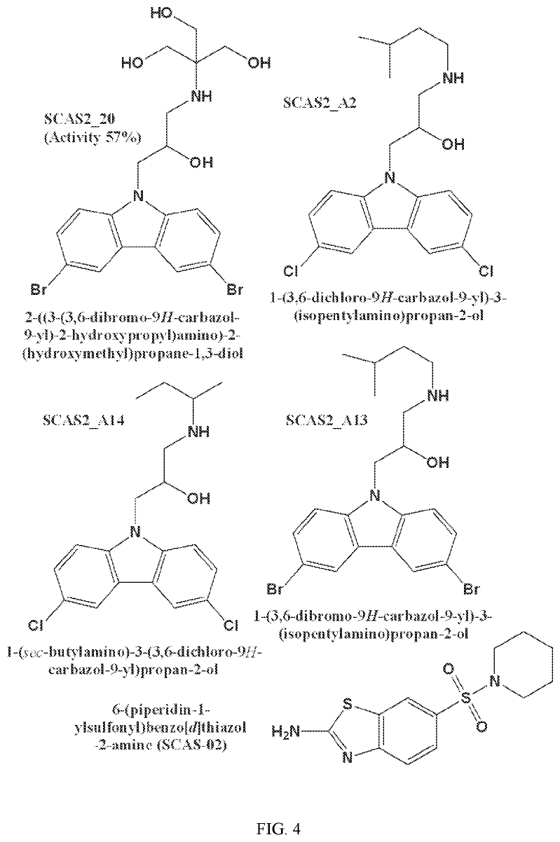

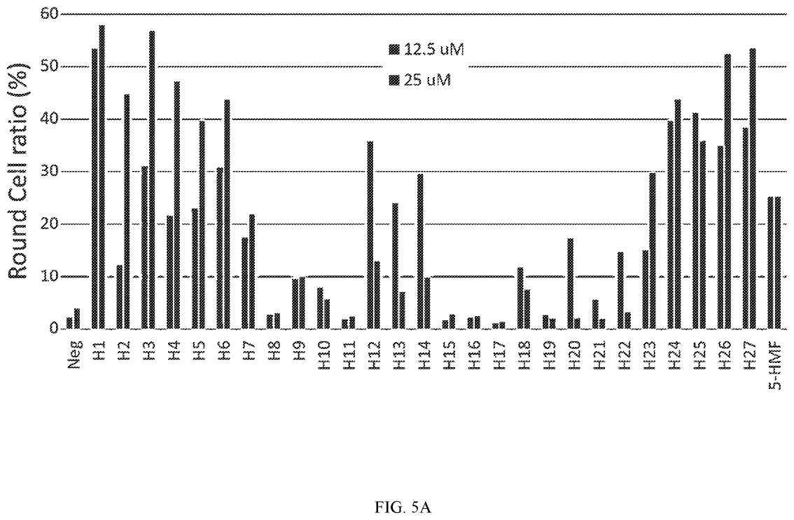

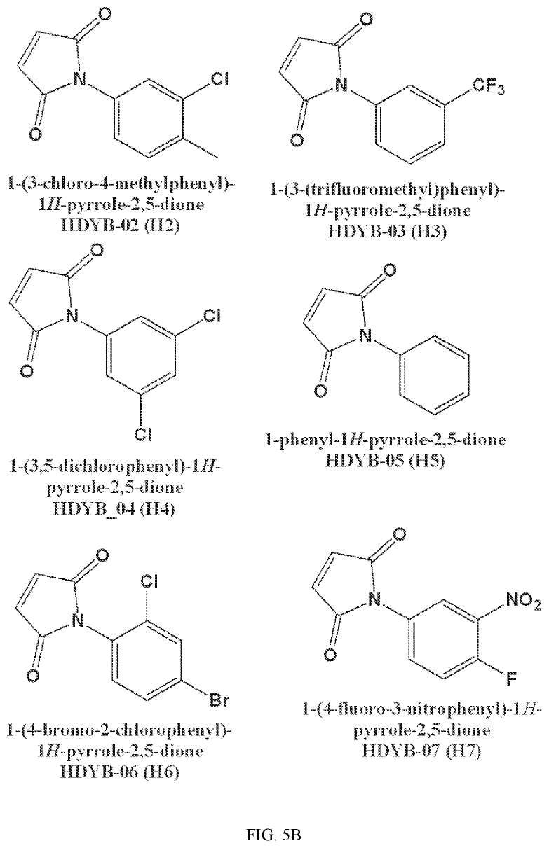

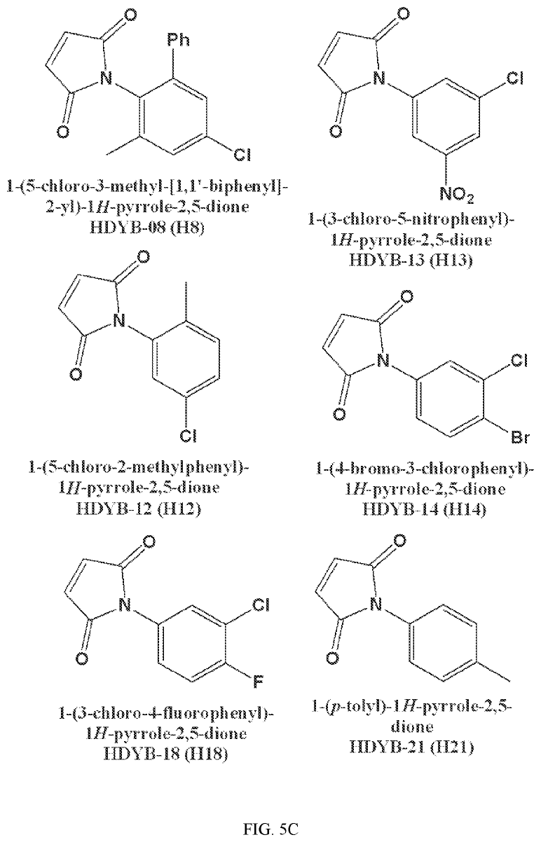

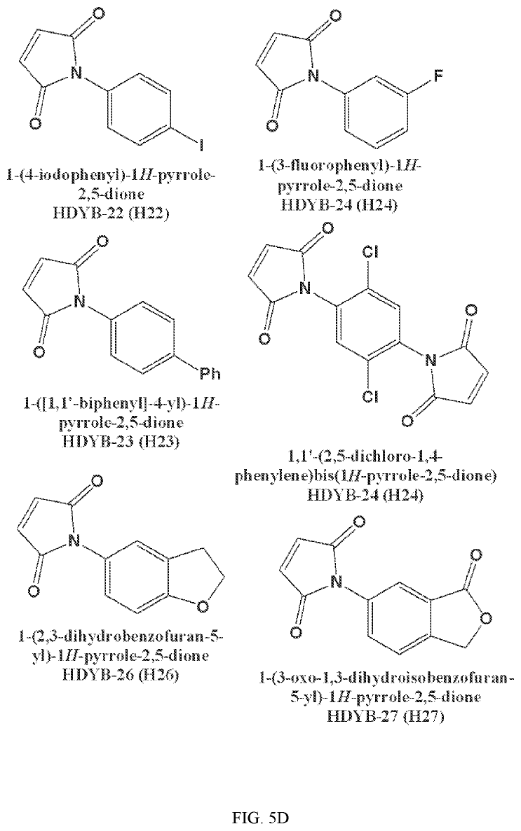

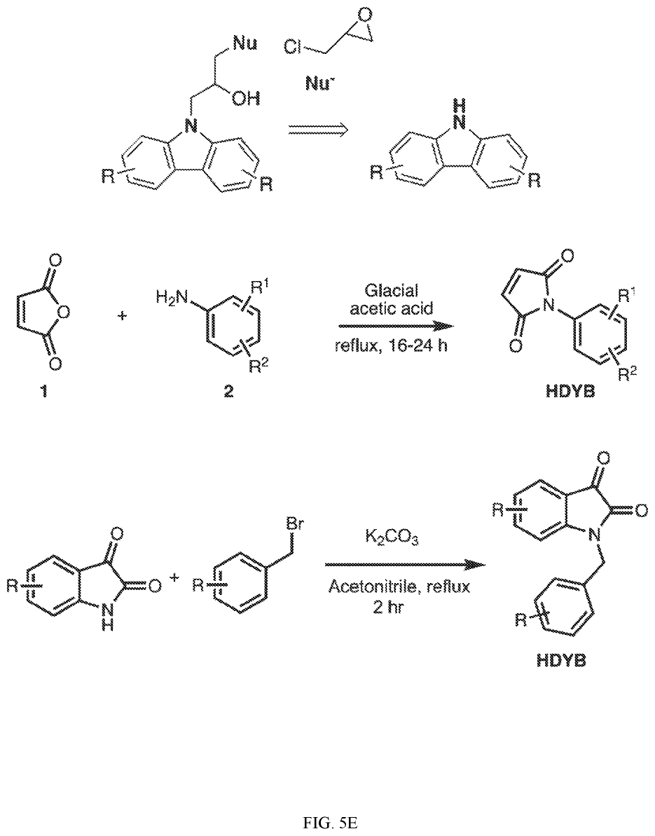

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure refers to an example and is not necessarily limited by such example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

“Subject” refers to any animal, preferably a human patient, livestock, horse, cow, pig, chicken, turkey, mouse, rodent, monkey, dog, cat, or other domestic pet.

As used herein, “alkyl” means a noncyclic straight chain or branched, unsaturated or saturated hydrocarbon such as those containing from 1 to 10 carbon atoms. The term “C₁-C₆ alkyl” refers to a linear or branched alkane derivative containing from 1 to 6, preferably from 1 to 3 (“C₁-C₃ alkyl”), carbon atoms and which is bound to the rest of the molecule through a single bond. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isoxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

“Alkylthio” refers to an alkyl group as defined above attached through a sulfur bridge. An example of an alkylthio is methylthio, (i.e., —S—CH₃).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above attached through an amino bridge. An example of an alkylamino is methylamino, (i.e., —NH—CH₃).

“Alkanoyl” refers to an alkyl as defined above attached through a carbonyl bridge (i.e., —(C═O)alkyl).

“Alkylsulfonyl” refers to an alkyl as defined above attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfinyl” refers to an alkyl as defined above with the indicated number of carbon atoms attached through a sulfinyl bridge (i.e. —S(═O)alkyl).

The terms “halogen” and “halo” refer to fluorine, chlorine, bromine, and iodine.

“Haloalkyl” refers to an alkyl group wherein one or more or all of the hydrogens are substituted with halogens, e.g., —CH₂CH₂Cl or —CF₃.

The term “sulfamoyl” refers to the amide of sulfonic acid (i.e., —S(═O)₂NRR′), examples include N-methylsulfamoyl, N-ethylsulfamoyl, N,N-dimethylsulfamoyl, N,N-diethylsulfamoyl, N-methyl-N-ethylsulfamoyl.

As one of ordinary skill in the art will recognize, combinations of substituents envisioned by this disclosure are those combinations that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein.

As used herein, the term “derivative” refers to a structurally similar compound that retains sufficient functional attributes of the identified analogue. The derivative may be structurally similar because it is lacking one or more atoms, contains an enriched atomic isotope, substituted, a salt, in different hydration/oxidation states, or because one or more atoms within the molecule are switched, such as, but not limited to, replacing an oxygen atom with a sulfur atom, or replacing an amino group with a hydroxy group. The derivative may be a prodrug. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze, hereby incorporated by reference.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol or acetamide, formamide, methanesulfonate, and benzamide derivatives of an amine functional group in the active compound and the like.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, alkylthio, nitro, cyano, OXO, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxy, alkyl, alkoxy, alkylthio, amino, alkylamino, dialkylamino, carbocyclyl, carbocycloalkyl, heterocarbocyclyl, heterocarbocycloalkyl, aryl, arylalkyl, heteroaryl, heteroarylalkyl.

As used herein, the terms “treat” and “treating” are not limited to the case where the subject (e.g. human patient) is cured and the disease is eradicated. Rather, embodiments, of the present disclosure also contemplate treatment that merely reduces symptoms, and/or delays disease progression.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the term “combination with” when used to describe administration of an agent with an additional treatment means such that the agent may be administered prior to, together with, or after the additional treatment, or a combination thereof, such that multiple agents are available at some overlapping time.

The term “effective amount” refers to that amount of a compound or pharmaceutical composition described herein that is sufficient to affect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the patient characteristics, particular compounds chosen, the dosing regimen to be followed, whether it is administered in combination with other agents, timing of administration, the tissue to which it is administered or targeted, and the physical delivery system in which it is carried.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

### Compounds for Managing Sickle Cell and Related Conditions

This disclosure relates to compounds for use in treating or preventing sickle cell disease or associated conditions. In certain embodiments, the compounds are 1-(thiazol-2-yl)urea derivatives or 1-phenyl-1H-pyrrole-2,5-dione derivatives.

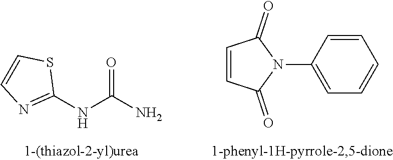 [SMILES: NC(=O)Nc1nccs1 ; O=C1C=CC(=O)N1c1ccccc1]

In certain embodiments, the compounds are 1-(9H-carbazol-9-yl)-3-mercaptopropan-2-ol derivatives.

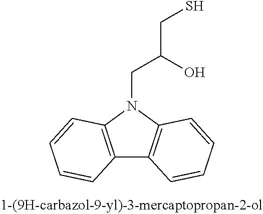 [SMILES: OC(CS)Cn1c2ccccc2c2ccccc21]

In certain embodiments, the compound has the following formula

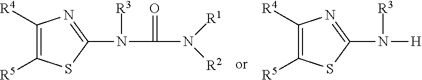

derivatives, prodrugs, or salts thereof wherein substituents are reported herein. In certain embodiments
R¹ is hydrogen, alkyl, halogenated alkyl, formyl, alkanoyl, benzyl, benzoyl, carbamoyl, sulfamoyl, carbocyclyl, aryl, or heterocyclyl, wherein R¹ is optionally substituted with one or more, the same or different, R¹⁰;
R² is hydrogen, alkyl, halogenated alkyl, formyl, alkanoyl, benzyl, benzoyl, carbamoyl, sulfamoyl, carbocyclyl, aryl, or heterocyclyl, wherein R² is optionally substituted with one or more, the same or different, R¹⁰; or
R¹ and R² and the attached nitrogen come together to form a carbocyclyl, aryl, or heterocyclyl optionally substituted, with one or more, the same or different, R¹⁰;
R³ is hydrogen, alkyl, halogenated alkyl, formyl, alkanoyl, benzyl, benzoyl, carbamoyl, sulfamoyl, carbocyclyl, aryl, or heterocyclyl;
R⁴ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁴ is optionally substituted with one or more, the same or different, R¹⁰;
R⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁵ is optionally substituted with one or more, the same or different, R¹⁰;
or R⁴ and R⁵ and the attached atoms come together to form an aryl, cyclic or heterocyclic ring optionally substituted with one or more, the same or different, R¹⁰.
R¹⁰ is alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, cyclopropylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, R¹ is an aryl or heterocyclyl optionally substituted. In certain embodiments, R¹ is phenyl disubstituted with substituents in the 2 position of the ring (ortho substituted) and the 5 position (meta substituted). In certain embodiments, R¹ is phenyl substituted with alkoxy in the 2 position (ortho substituted). In certain embodiments, R¹ is phenyl substituted with a halogen in the 5 position (meta substituted).

In certain embodiments, R⁴ and R⁵ and the attached atoms come together to form a cyclic or heterocyclic ring optionally substituted with one or more, the same or different, R¹⁰.

In certain embodiments, R⁴ and R⁵ and the attached atoms come together to form aryl or phenyl ring optionally substituted with one or more, the same or different, R¹⁰.

In certain embodiments, R¹ is phenyl disubstituted with substituents in the 2 position (ortho substituted), a halogen in the 5 position (meta substituted) and R² is hydrogen or alkyl. In certain embodiments, R¹ is phenyl substituted with alkoxy in the 2 position (ortho substituted) and R² is hydrogen or alkyl. In certain embodiments, R¹ is phenyl substituted a halogen in the 5 position (meta substituted) and R² is hydrogen or alkyl.

In certain embodiments, R¹ is phenyl disubstituted with substituents in the 2 position (ortho substituted), a halogen in the 5 position (meta substituted) and R² is hydrogen. In certain embodiments, R¹ is phenyl substituted with alkoxy in the 2 position (ortho substituted) and R² is hydrogen. In certain embodiments, R¹ is phenyl substituted a halogen in the 5 position (meta substituted) and R² is hydrogen.

In certain embodiments, R¹ is phenyl disubstituted with substituents in the 2 position (ortho substituted), a halogen in the 5 position (meta substituted) and R² is hydrogen or alkyl and R³ is hydrogen or alkyl. In certain embodiments, R¹ is phenyl substituted with alkoxy in the 2 position (ortho substituted) and R² is hydrogen or alkyl and R³ is hydrogen or alkyl. In certain embodiments, R¹ is phenyl substituted a halogen in the 5 position (meta substituted) and R² is hydrogen or alkyl and R³ is hydrogen or alkyl.

In certain embodiments, R¹ is phenyl disubstituted with substituents in the 2 position (ortho substituted), a halogen in the 5 position (meta substituted) and R² is hydrogen and R³ is hydrogen or alkyl. In certain embodiments, R¹ is phenyl substituted with alkoxy in the 2 position (ortho substituted) and R² is hydrogen and R³ is hydrogen or alkyl. In certain embodiments, R¹ is phenyl substituted a halogen in the 5 position (meta substituted) and R² is hydrogen and R³ is hydrogen or alkyl.

In certain embodiments, the compound has the following formula

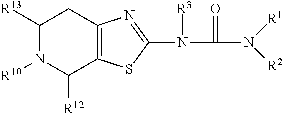

derivatives, prodrugs, or salts thereof wherein substituents are reported herein. In certain embodiments
R¹ is hydrogen, alkyl, halogenated alkyl, formyl, alkanoyl, benzyl, benzoyl, carbamoyl, sulfamoyl, carbocyclyl, aryl, or heterocyclyl, wherein R¹ is optionally substituted with one or more, the same or different, R¹⁰;
R² is hydrogen, alkyl, halogenated alkyl, formyl, alkanoyl, benzyl, benzoyl, carbamoyl, sulfamoyl, carbocyclyl, aryl, or heterocyclyl, wherein R² is optionally substituted with one or more, the same or different, R¹⁰; or
R¹ and R² and the attached nitrogen come together to form a carbocyclyl, aryl, or heterocyclyl optionally substituted, with one or more, the same or different, R¹⁰;
R¹⁰ is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R¹⁰ is optionally substituted with one or more, the same or different, R¹¹;
R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, cyclopropylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;
R¹² is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl; and
R¹³ is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl.

In certain embodiments, R¹ is an aryl or heterocyclyl optionally substituted. In certain embodiments, R¹ is phenyl disubstituted with substituents in the 2 position of the ring (ortho substituted) and a halogen in the 5 position (meta substituted). In certain embodiments, R¹ is phenyl substituted with alkoxy in the 2 position (ortho substituted). In certain embodiments, R¹ is phenyl substituted a halogen in the 5 position (meta substituted).

In certain embodiments, R¹ is phenyl disubstituted with substituents in the 2 position (ortho substituted), a halogen in the 5 position (meta substituted), and R² is hydrogen. In certain embodiments, R¹ is phenyl substituted with alkoxy in the 2 position (ortho substituted) and R² is hydrogen. In certain embodiments, R¹ is phenyl substituted with substituents in the 5 position (meta substituted) with a halogen and R² is hydrogen.

In certain embodiments, R¹ is phenyl disubstituted with substituents in the 2 position (ortho substituted), a halogen in the 5 position (meta substituted) and R² is hydrogen and R³ is hydrogen, and R¹⁰ is sulfamoyl, alkylsulfonyl, or carbocyclylsulfonyl. In certain embodiments, R¹ is phenyl substituted with alkoxy in the 2 position (ortho substituted) and R² is hydrogen and R³ is hydrogen and R¹⁰ is sulfamoyl, alkylsulfonyl, or carbocyclylsulfonyl.

In certain embodiments, R¹ is phenyl disubstituted with substituents in the 2 position (ortho substituted), a halogen in the 5 position (meta substituted) and R² is hydrogen and R³ is hydrogen, and R¹⁰ is sulfamoyl, alkylsulfonyl, or carbocyclylsulfonyl, and R¹² and R¹³ are hydrogen. In certain embodiments, R¹ is phenyl substituted with alkoxy in the 2 position (ortho substituted) and R² is hydrogen and R³ is hydrogen and R¹⁰ is sulfamoyl, alkylsulfonyl, or carbocyclylsulfonyl, and R¹² and R¹³ are hydrogen.

In certain embodiments, the compound has the following formula

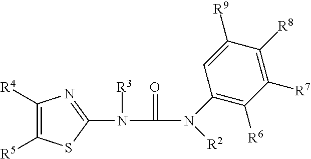

derivatives, prodrugs, or salts thereof wherein substituents are reported herein. In certain embodiments
R² is hydrogen, alkyl, halogenated alkyl, formyl, alkanoyl, benzyl, benzoyl, carbamoyl, sulfamoyl, carbocyclyl, aryl, or heterocyclyl, wherein R² is optionally substituted with one or more, the same or different, R¹⁰; or
R³ is hydrogen, alkyl, halogenated alkyl, formyl, alkanoyl, benzyl, benzoyl, carbamoyl, sulfamoyl, carbocyclyl, aryl, or heterocyclyl;
R⁴ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁴ is optionally substituted with one or more, the same or different, R¹⁰;
R⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁵ is optionally substituted with one or more, the same or different, R¹⁰;
R⁶ is hydrogen, alkyl, halogenated alkyl, halogen, hydroxy, alkoxy, alkylthio, amino, sulfamoyl, carbocyclyl, aryl, or heterocyclyl, wherein R⁶ is optionally substituted with one or more, the same or different, R¹⁰;
R⁷ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁷ is optionally substituted with one or more, the same or different, R¹⁰;
R⁸ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁸ is optionally substituted with one or more, the same or different, R¹⁰;
R⁹ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁹ is optionally substituted with one or more, the same or different, R¹⁰;
R¹⁰ is alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, methylsulfinyl, ethylsulfinyl, mesyl, ethylsulfonyl, cyclopropylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, R⁹ is halogen and R⁶ is alkoxy. In certain embodiments, R⁷ and R⁸ are hydrogen, and R⁹ is halogen and R⁶ is alkoxy. In certain embodiments, R⁹ is halogen and R⁶ is alkoxy. In certain embodiments, R⁷ and R⁸ are hydrogen, and R⁹ is halogen and R⁶ is alkoxy, and R² and R³ are hydrogen.

In certain embodiments, the compound has the following formula

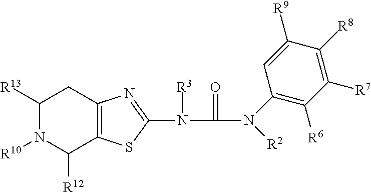

derivatives, prodrugs, or salts thereof wherein substituents are reported herein. In certain embodiments
R² is hydrogen, alkyl, halogenated alkyl, formyl, alkanoyl, benzyl, benzoyl, carbamoyl, sulfamoyl, carbocyclyl, aryl, or heterocyclyl, wherein R² is optionally substituted with one or more, the same or different, R¹⁰; or
R³ is hydrogen, alkyl, halogenated alkyl, formyl, alkanoyl, benzyl, benzoyl, carbamoyl, sulfamoyl, carbocyclyl, aryl, or heterocyclyl;
R⁶ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁶ is optionally substituted with one or more, the same or different, R¹⁰;
R⁷ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁷ is optionally substituted with one or more, the same or different, R¹⁰;
R⁸ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁸ is optionally substituted with one or more, the same or different, R¹⁰;
R⁹ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁹ is optionally substituted with one or more, the same or different, R¹⁰;
R¹⁰ is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, cyclopropylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;
R¹² is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl; and
R¹³ is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl.

In certain embodiments, the compound is 2-(3-(5-chloro-2-methoxyphenyl)ureido)-N,N-dimethyl-6,7-dihydrothiazolo[5,4-c]pyridine-5 (4H)-sulfonamide (SCAS2_01), derivative, prodrug, or salt thereof.

In certain embodiments, the compound is 1-(5-chloro-2-methoxyphenyl)-3-(5-(cyclopropylsulfonyl)-4,5,6,7-tetrahydrothiazolo[5,4-c]pyridin-2-yl)urea (SCAS2_05) derivative, prodrug, or salt thereof.

In certain embodiments, this disclosure relates to a compound 6-(piperidin-1-ylsulfonyl)benzo[d]thiazol-2-amine (SCAS-02), derivative, prodrug, or salt thereof.

In certain embodiments, the compound has the following formula

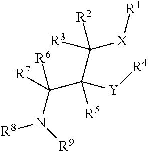

derivatives, prodrugs, or salts thereof wherein substituents are reported herein. In certain embodiments
X is S or O; or X is S, NH, or O;
Y is O or S;
R¹ is hydrogen, alkyl, halogenated alkyl, formyl, alkanoyl, benzyl, benzoyl, carbamoyl, carbocyclyl, aryl, or heterocyclyl, wherein R¹ is optionally substituted with one or more, the same or different, R²⁰; or
R² is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R² is optionally substituted with one or more, the same or different, R²⁰;
R³ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R³ is optionally substituted with one or more, the same or different, R²⁰;
R⁴ is hydrogen, alkyl, halogenated alkyl, formyl, alkanoyl, benzyl, benzoyl, carbamoyl, carbocyclyl, aryl, or heterocyclyl, wherein R⁴ is optionally substituted with one or more, the same or different, R²⁰;
or R¹ and R⁴ and the attached atoms come together to form a heterocyclic ring, optionally substituted with one or more, the same or different, R²⁰;
R⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁵ is optionally substituted with one or more, the same or different, R²⁰;
R⁶ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁶ is optionally substituted with one or more, the same or different, R²⁰;
R⁷ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁷ is optionally substituted with one or more, the same or different, R²⁰;
R⁸ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁸ is optionally substituted with one or more, the same or different, R²⁰;
R⁹ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R′ is optionally substituted with one or more, the same or different, R²⁰;
or R⁸ and R⁹ and the attached atoms come together to form a heterocyclic ring, optionally substituted with one or more, the same or different, R²⁰;
R²⁰ is alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R²⁰ is optionally substituted with one or more, the same or different, R²¹; and
R²¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, X is S and Y is O. In certain embodiments, X is S and R¹ is an alkyl or tertiary alkyl optionally substituted with a substituent. In certain embodiments, Y is O and R⁴ is hydrogen optionally substituted with a substituent. In certain embodiments, R², R³, R⁵, R⁶ and R⁷ are hydrogen.

In certain embodiments, the compound has the following formula

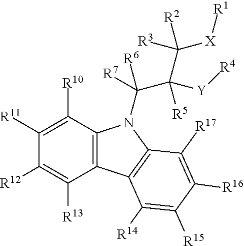

derivatives, prodrugs, or salts thereof wherein substituents are reported herein. In certain embodiments
X is S or O; or X is S, NH, or O;
Y is O or S;
R¹ is hydrogen, alkyl, halogenated alkyl, formyl, alkanoyl, benzyl, benzoyl, carbamoyl, carbocyclyl, aryl, or heterocyclyl, wherein R¹ is optionally substituted with one or more, the same or different, R²⁰; or
R² is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R² is optionally substituted with one or more, the same or different, R²⁰;
R³ is hydrogen, alkyl, halogenated alkyl, halogen, hydroxy, alkoxy, alkylthio, amino, carbocyclyl, aryl, or heterocyclyl, wherein R³ is optionally substituted with one or more, the same or different, R²⁰;
R⁴ is hydrogen, alkyl, halogenated alkyl, formyl, alkanoyl, benzyl, benzoyl, carbamoyl, carbocyclyl, aryl, or heterocyclyl, wherein R⁴ is optionally substituted with one or more, the same or different, R²⁰;
or R¹ and R⁴ and the attached atoms come together to form a heterocyclic ring, optionally substituted with one or more, the same or different, R²⁰;
R⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁵ is optionally substituted with one or more, the same or different, R²⁰;
R⁶ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁶ is optionally substituted with one or more, the same or different, R²⁰;
R⁷ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁷ is optionally substituted with one or more, the same or different, R²⁰;
R¹⁰ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹⁰ is optionally substituted with one or more, the same or different, R²⁰; or
R¹¹ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹¹ is optionally substituted with one or more, the same or different, R²⁰;
R¹² is hydrogen, alkyl, halogenated alkyl, halogen, hydroxy, alkoxy, alkylthio, amino, carbocyclyl, aryl, or heterocyclyl, wherein R¹² is optionally substituted with one or more, the same or different, R²⁰;
R¹³ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹³ is optionally substituted with one or more, the same or different, R²⁰;
R¹⁴ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹⁴ is optionally substituted with one or more, the same or different, R²⁰;
R¹⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹⁵ is optionally substituted with one or more, the same or different, R²⁰;
R¹⁶ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹⁶ is optionally substituted with one or more, the same or different, R²⁰;
R¹⁷ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹⁷ is optionally substituted with one or more, the same or different, R²⁰;
R²⁰ is alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R²⁰ is optionally substituted with one or more, the same or different, R²¹; and
R²¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, X is S and Y is O, and R¹² and R¹⁵ are halogen. In certain embodiments, X is S and R¹ is an alkyl or tertiary alkyl optionally substituted with a substituent and R¹² and R¹⁵ are halogen. In certain embodiments, Y is O and R⁴ is hydrogen optionally substituted with a substituent and R¹² and R¹⁵ are halogen. In certain embodiments, R², R³, R⁵, R⁶ and R⁷ are hydrogen and R¹² and R¹⁵ are halogen. In certain embodiments, R¹⁰, R¹¹, R¹³, R¹⁴, R¹⁶, and R¹⁷ are hydrogen.

In certain embodiments, X is NH and Y is O, and R¹² and R¹⁵ are halogen. In certain embodiments, X is S and R¹ is an alkyl or tertiary alkyl optionally substituted with a substituent and R¹² and R¹⁵ are halogen. In certain embodiments, Y is O and R⁴ is hydrogen optionally substituted with a substituent and R¹² and R¹⁵ are halogen. In certain embodiments, R², R³, R⁵, R⁶ and R⁷ are hydrogen and R¹² and R¹⁵ are halogen. In certain embodiments, R¹⁰, R¹¹, R¹³, R¹⁴, R¹⁶, and R¹⁷ are hydrogen.

In certain embodiments the compound is 1-(tert-butylthio)-3-(3,6-dichloro-9H-carbazol-9-yl) propan-2-ol (SCAS2_61), derivatives, prodrugs, or salts thereof.

In certain embodiments, the compound has the following formula

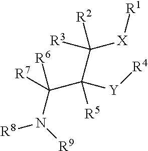

derivatives, prodrugs, or salts thereof wherein substituents are reported herein. In certain embodiments
X is S, NH, or O;
Y is O or S;
R¹ is hydrogen, alkyl, halogenated alkyl, formyl, alkanoyl, benzyl, benzoyl, carbamoyl, carbocyclyl, aryl, or heterocyclyl, wherein R¹ is optionally substituted with one or more, the same or different, R²⁰; or
R² is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R² is optionally substituted with one or more, the same or different, R²⁰;
R³ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R³ is optionally substituted with one or more, the same or different, R²⁰;
R⁴ is hydrogen, alkyl, halogenated alkyl, formyl, alkanoyl, benzyl, benzoyl, carbamoyl, carbocyclyl, aryl, or heterocyclyl, wherein R⁴ is optionally substituted with one or more, the same or different, R²⁰;
or R¹ and R⁴ and the attached atoms come together to form a heterocyclic ring, optionally substituted with one or more, the same or different, R²⁰;
R⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁵ is optionally substituted with one or more, the same or different, R²⁰;
R⁶ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁶ is optionally substituted with one or more, the same or different, R²⁰;
R⁷ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁷ is optionally substituted with one or more, the same or different, R²⁰;
R⁸ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁸ is optionally substituted with one or more, the same or different, R²⁰;
R⁹ is hydrogen, alkyl, halogenated alkyl, halogen, hydroxy, alkoxy, alkylthio, amino, carbocyclyl, aryl, or heterocyclyl, wherein R⁹ is optionally substituted with one or more, the same or different, R²⁰;
or R⁸ and R⁹ and the attached atoms come together to form a heterocyclic ring, optionally substituted with one or more, the same or different, R²⁰;
R²⁰ is alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R²⁰ is optionally substituted with one or more, the same or different, R²¹; and
R²¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, cyclopropylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, X is S and Y is O. In certain embodiments, X is S and R¹ is an alkyl or tertiary alkyl optionally substituted with a substituent. In certain embodiments, Y is O and R⁴ is hydrogen optionally substituted with a substituent. In certain embodiments, R², R³, R⁵, R⁶ and R⁷ are hydrogen.

In certain embodiments, the compound has the following formula

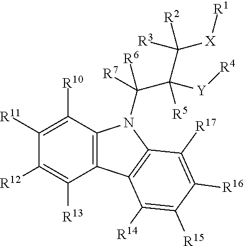

derivatives, prodrugs, or salts thereof wherein substituents are reported herein. In certain embodiments
X is S or O; or S, NH, or O;
Y is O or S;
R¹ is hydrogen, alkyl, halogenated alkyl, formyl, alkanoyl, benzyl, benzoyl, carbamoyl, carbocyclyl, aryl, or heterocyclyl, wherein R¹ is optionally substituted with one or more, the same or different, R²⁰; or
R² is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R² is optionally substituted with one or more, the same or different, R²⁰;
R³ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R³ is optionally substituted with one or more, the same or different, R²⁰;
R⁴ is hydrogen, alkyl, halogenated alkyl, formyl, alkanoyl, benzyl, benzoyl, carbamoyl, carbocyclyl, aryl, or heterocyclyl, wherein R⁴ is optionally substituted with one or more, the same or different, R²⁰; or R¹ and R⁴ and the attached atoms come together to form a heterocyclic ring, optionally substituted with one or more, the same or different, R²⁰;
R⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁵ is optionally substituted with one or more, the same or different, R²⁰;
R⁶ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁶ is optionally substituted with one or more, the same or different, R²⁰;
R⁷ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R⁷ is optionally substituted with one or more, the same or different, R²⁰;
R¹⁰ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹⁰ is optionally substituted with one or more, the same or different, R²⁰; or
R¹¹ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹¹ is optionally substituted with one or more, the same or different, R²⁰;
R¹² is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹² is optionally substituted with one or more, the same or different, R²⁰;
R¹³ is hydrogen, alkyl, halogenated alkyl, halogen, hydroxy, alkoxy, alkylthio, amino, carbocyclyl, aryl, or heterocyclyl, wherein R¹³ is optionally substituted with one or more, the same or different, R²⁰;
R¹⁴ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹⁴ is optionally substituted with one or more, the same or different, R²⁰;
R¹⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹⁵ is optionally substituted with one or more, the same or different, R²⁰;
R¹⁶ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹⁶ is optionally substituted with one or more, the same or different, R²⁰;
R¹⁷ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, carbocyclyl, aryl, or heterocyclyl, wherein R¹⁷ is optionally substituted with one or more, the same or different, R²⁰;
R²⁰ is alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R²⁰ is optionally substituted with one or more, the same or different, R²¹; and
R²¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, cyclopropylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, X is S and Y is O, and R¹² and R¹⁵ are halogen.

In certain embodiments, X is NH and Y is O, and R¹² and R¹⁵ are halogen.

In certain embodiments, X is S or NH, and R¹ is an alkyl or tertiary alkyl optionally substituted with a substituent and R¹² and R¹⁵ are halogen. In certain embodiments, Y is O and R⁴ is hydrogen optionally substituted with a substituent and R¹² and R¹⁵ are halogen. In certain embodiments, R², R³, R⁵, R⁶ and R⁷ are hydrogen and R¹² and R¹⁵ are halogen. In certain embodiments, R¹⁰, R¹¹, R¹³, R¹⁴, R¹⁶, and R¹⁷ are hydrogen.

In certain embodiments, the compound is 2-((3-(3,6-dibromo-9H-carbazol-9-yl)-2-hydroxypropyl)amino)-2-(hydroxymethyl) propane-1,3-diol (SCAS2_20), derivative, prodrug, or salt thereof.

In certain embodiments, the compound is 1-(tert-butylthio)-3-(3,6-dichloro-9H-carbazol-9-yl) propan-2-ol (SCAS2_61), derivative, prodrug, or salt thereof.

In certain embodiments, the compound is 1-(3,6-dichloro-9H-carbazol-9-yl)-3-(isopentylamino) propan-2-ol (SCAS2_A2), derivative, prodrug, or salt thereof.

In certain embodiments, the compound is 1-(3,6-dibromo-9H-carbazol-9-yl)-3-(isopentylamino) propan-2-ol (SCAS2_A13), derivative, prodrug, or salt thereof.

In certain embodiments, the compound is 1-(sec-butylamino)-3-(3,6-dichloro-9H-carbazol-9-yl) propan-2-ol (SCAS2_A14), derivative, prodrug, or salt thereof.

In certain embodiments, the compound has the following formula

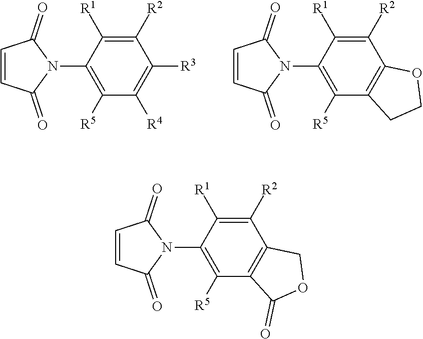

derivatives, prodrugs, or salts thereof wherein substituents are reported herein. In certain embodiments
R¹ is hydrogen, alkyl, halogenated alkyl, formyl, alkanoyl, benzyl, benzoyl, carbamoyl, sulfamoyl, carbocyclyl, aryl, or heterocyclyl, wherein R¹ is optionally substituted with one or more, the same or different, R¹⁰;
R² is hydrogen, alkyl, halogenated alkyl, formyl, alkanoyl, benzyl, benzoyl, carbamoyl, sulfamoyl, carbocyclyl, aryl, or heterocyclyl, wherein R² is optionally substituted with one or more, the same or different, R¹⁰; or
R³ is hydrogen, alkyl, halogenated alkyl, formyl, alkanoyl, benzyl, benzoyl, carbamoyl, sulfamoyl, carbocyclyl, aryl, or heterocyclyl; or
R⁴ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁴ is optionally substituted with one or more, the same or different, R¹⁰; or
R³ and R⁴ and the attached nitrogen come together to form a carbocyclyl, aryl, or heterocyclyl optionally substituted, with one or more, the same or different, R¹⁰;
R⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁵ is optionally substituted with one or more, the same or different, R¹⁰;
R¹⁰ is alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, the compound is 1-(3-chlorophenyl)-1H-pyrrole-2,5-dione (HDYB-1) derivative, prodrug, or salt thereof.

In certain embodiments, the compound is 1-(3-chloro-4-methylphenyl)-1H-pyrrole-2,5-dione (HDYB-2) derivative, prodrug, or salt thereof.

In certain embodiments, the compound is 1-(3-(trifluoromethyl)phenyl)-1H-pyrrole-2,5-dione (HDYB-3) derivative, prodrug, or salt thereof.

In certain embodiments, the compound is 1-(3,5-dichlorophenyl)-1H-pyrrole-2,5-dione (HDYB-4) derivative, prodrug, or salt thereof.

In certain embodiments, the compound is 1-phenyl-1H-pyrrole-2,5-dione (HDYB-5) derivative, prodrug, or salt thereof.

In certain embodiments, the compound is 1-(4-bromo-2-chlorophenyl)-1H-pyrrole-2,5-dione (HDYB-6) derivative, prodrug, or salt thereof.

In certain embodiments, the compound is 1-(4-fluoro-3-methylphenyl)-1H-pyrrole-2,5-dione (HDYB-7) derivative, prodrug, or salt thereof.

In certain embodiments, the compound is 1-(5-chloro-3-methyl-[1,1′-biphenyl]-2-yl)-1H-pyrrole-2,5-dione (HDYB-8) derivative, prodrug, or salt thereof.

In certain embodiments, the compound is 1-(5-chloro-2-methylphenyl)-1H-pyrrole-2,5-dione (HDYB-12) derivative, prodrug, or salt thereof.

In certain embodiments, the compound is 1-(3-chloro-5-nitrophenyl)-1H-pyrrole-2,5-dione (HDYB-13) derivative, prodrug, or salt thereof.

In certain embodiments, the compound is 1-(4-bromo-3-chlorophenyl)-1H-pyrrole-2,5-dione (HDYB-14) derivative, prodrug, or salt thereof.

In certain embodiments, the compound is 1-(3-chloro-4-fluorophenyl)-1H-pyrrole-2,5-dione (HDYB-18) derivative, prodrug, or salt thereof.

In certain embodiments, the compound is 1-(p-tolyl)-1H-pyrrole-2,5-dione (HDYB-21) derivative, prodrug, or salt thereof.

In certain embodiments, the compound is 1-(4-iodophenyl)-1H-pyrrole-2,5-dione (HDYB-22) derivative, prodrug, or salt thereof.

In certain embodiments, the compound is 1-([1,1′-biphenyl]-4-yl)-1H-pyrrole-2,5-dione (HDYB-23) derivative, prodrug, or salt thereof.

In certain embodiments, the compound is 1-(3-fluorophenyl)-1H-pyrrole-2,5-dione (HDYB-24) derivative, prodrug, or salt thereof.

In certain embodiments, the compound is 1-(2,3-dihydrobenzofuran-5-yl)-1H-pyrrole-2,5-dione (HDYB-26) derivative, prodrug, or salt thereof.

In certain embodiments, the compound is 1-(3-oxo-1,3-dihydroisobenzofuran-5-yl)-1H-pyrrole-2,5-dione (HDYB-27) derivative, prodrug, or salt thereof.

In certain embodiments, the compound is 1,1′-(2,5-dichloro-1,4-phenylene)bis(1H-pyrrole-2,5-dione) (HDYB-25) derivative, prodrug, or salt thereof.

### Methods of Managing Sickle Cell Disease and Conditions Related Thereto

In certain embodiments, this disclosure contemplates using compounds disclosed herein as modulators of hemoglobin, e.g., treating disorders mediated by hemoglobin and disorders that would benefit from tissue and/or cellular oxygenation. Sickle cell disease is a group of disorders that affects hemoglobin. People with this disorder have atypical hemoglobin molecules called hemoglobin S, which can distort red blood cells into a sickle, or crescent, shape. When red blood cells sickle, they break down prematurely, which can lead to anemia. Anemia can cause shortness of breath, fatigue, and delayed growth and development in children. In certain embodiments, this disclosure relates to method of treating anemia, shortness of breath, fatigue, and delayed growth and development in children by administering a compound disclosed herein to a subject in need thereof.

In certain embodiments, the compound disclosed herein may be used in methods disclosed herein in combination with other agents used to treat or prevent sickle cell disease, conditions, or complications. In certain embodiments, the subject to be treated is diagnosed with a disease or condition reported herein.

In certain embodiments, this disclosure relates to using a compound disclosed herein for treating sickle cell disease resulting in fewer, or less severe side effects, greater efficacy, greater response rate, or combinations thereof compared to existing therapies such as hydroxyurea or another therapeutic agent.

In certain embodiments, the compound disclosed herein may be used in methods of treating sickle cell diseases including sickle cell anemia, sickle-hemoglobin C disease, sickle beta-plus-thalassemia and sickle beta-zero-thalassemia.

In certain embodiments, the compound disclosed herein may be used in methods of treating hematological disorders such as sickle cell disease, pulmonary disease such as idiopathic pulmonary fibrosis, and hypoxia.

In certain embodiments, the compound disclosed herein may be used in methods of increasing affinity of hemoglobin for oxygen in a patient.

For purposes of treatment of sickle cell disease, beneficial or desired clinical results include, but are not limited to, multi-lineage hematologic improvement, decrease in the number of required blood transfusions, decrease in infections, decreased bleeding, and the like. For purposes of treatment of interstitial pulmonary fibrosis, beneficial or desired clinical results include, but are not limited to, reduction in hypoxia, reduction in fibrosis, and the like.

Sickle cell trait or the carrier state is the heterozygous form characterized by the presence of HbS, absence of anemia, inability to concentrate urine (isosthenuria), and hematuria. Under conditions leading to hypoxia, it may become a pathologic risk factor. Accordingly, in some embodiments, the disclosed compounds disclosed herein are used to treat a subject heterozygous for an autosomal recessive mutation in the beta-chain gene of hemoglobin (i.e., heterozygous for HbS).

In certain embodiments, this disclosure relates to methods of treating thalassemia, for example beta-thalassemia, in a subject in need thereof by administering to the subject an effective amount of a compound disclosed herein optionally in combination with hydroxyurea or another therapeutic agent.

Beta-thalassemias are a group of inherited blood disorders caused by a variety of mutational mechanisms that result in a reduction or absence of synthesis of beta-globin and leading to accumulation of aggregates of unpaired, insoluble alpha-chains that cause ineffective erythropoiesis, accelerated red cell destruction, and severe anemia. Subjects with beta-thalassemia exhibit variable phenotypes ranging from severe anemia to clinically asymptomatic individuals. The mutations can involve a single base substitution or deletions or inserts within, near or upstream of the beta globin gene. For example, mutations occurring in the promoter regions preceding the beta-globin genes cause production of abnormal splice variants.

Examples of a thalassemia contemplated for treatment with compounds disclosed herein optionally in combination with hydroxyurea or another therapeutic agent include thalassemia minor, thalassemia intermedia, and thalassemia major.

Thalassemia minor refers to thalassemia where only one of beta-globin alleles bears a mutation. Individuals typically suffer from microcytic anemia. Detection usually involves lower than normal MCV value (<80 fL) plus an increase in fraction of Hemoglobin A2 (>3.5%) and a decrease in fraction of Hemoglobin A (<97.5%).

Thalassemia intermedia refers to a thalassemia intermediate between the major and minor forms. Affected individuals can often manage a normal life but may need occasional transfusions, e.g., at times of illness or pregnancy, depending on the severity of their anemia.

Thalassemia major refers to a thalassemia where both beta-globin alleles have thalassemia mutations. If left untreated, it causes anemia, splenomegaly, and severe bone deformities and typically leads to death before age 20. Treatment consists of periodic blood transfusion; splenectomy if splenomegaly is present, and treatment of transfusion-caused iron overload.

Although carriers of sickle cell trait typically do not suffer from sickle cell disease, individuals with one copy of HbS and one copy of a gene that codes for another abnormal variant of hemoglobin, such as HbC or Hb beta-thalassemia, have a less severe form of the disease. For example, another specific defect in beta-globin causes another structural variant, hemoglobin C (HbC). Hemoglobin C (abbreviated as HbC) is an abnormal hemoglobin in which substitution of a glutamic acid residue with a lysine residue at the 6th position of the beta-globin chain has occurred. A subject that is a double heterozygote for HbS and HbC (HbSC disease) is typically characterized by symptoms of moderate clinical severity. In certain embodiments, this disclosure contemplates treatment by administering effective amounts of compounds disclosed herein to subject that is a double heterozygote for HbS and HbC (HbSC disease).

Another structural variant of beta-globin is hemoglobin E or hemoglobin E (HbE). HbE is an abnormal hemoglobin in which substitution of a glutamic acid residue with a lysine residue at the 26th position of the beta-globin chain has occurred. A subject that is a double heterozygote for HbS and HbE has HbS/HbE syndrome. In certain embodiments, this disclosure contemplates treatment by administering effective amounts of a compound disclosed herein to subject that is a double heterozygote for HbS and HbE (HbS/HbE) syndrome.

Some mutations in the beta-globin gene can cause other structural variations of hemoglobin or can cause a deficiency in the amount of beta-globin being produced. These types of mutations are referred to as beta-thalassemia mutations. In certain embodiments, this disclosure contemplates treatment by administering effective amounts of a compound disclosed herein to subject that is diagnosed with as beta-thalassemia mutations.

The absence of beta-globin is referred to as beta-zero (beta-0) thalassemia. A subject that is a double heterozygote for HbS and beta-0 thalassemia (i.e., HbS/beta-0 thalassemia) can suffer symptoms clinically similar to sickle cell anemia. In certain embodiments, this disclosure contemplates treatment by administering effective amounts of a compound disclosed herein to subject that is diagnosed with beta-zero thalassemia.

A reduced amount of beta-globin is referred to as beta-plus (beta+) thalassemia. A subject that is a double heterozygote for HbS and beta+thalassemia (i.e., HbS/beta+thalassemia) can have mild-to-moderate severity of clinical symptoms with variability among different ethnicities. In certain embodiments, this disclosure contemplates treatment by administering effective amounts of a compound disclosed herein to subject that is diagnosed with beta-plus thalassemia.

In certain embodiments, this disclosure contemplates treating retinopathy complications due to sickle cell diseases or complications by administering an effective amount of a compound disclosed herein, optionally in combination with hydroxyurea or another therapeutic agent. Sickle retinopathy occurs when the retinal blood vessels get occluded by sickle red blood cells and the retina becomes ischemic. In sickle cell disease, this occurs mostly in the peripheral retina, which does not obscure vision at first. Eventually, the entire peripheral retina of the sickle cell patient becomes occluded and many neovascular formations occur. In certain embodiments, this disclosure contemplates administration of a compound disclosed herein optionally in combination with hydroxyurea or another therapeutic agent in order to reduce or inhibit the formation of occlusions in the peripheral retina of a sickle cell patient.

In some embodiments, a compound disclosed herein is used to increase hemoglobin expression or activity in non-erythroid cells including, but not limited to, macrophage, retinal pigment cells, and alveolar epithelial cells such as alveolar type II (ATII) cells and Clara cells. In some embodiments, a compound disclosed herein is used to increase hemoglobin expression or activity in non-erythroid cells at interfaces where oxygen-carbon dioxide diffusion occurs, including, but not limited to the eyes and lungs. In some embodiments, a compound disclosed herein is used to induce, increase, or enhance hemoglobin synthesis or activity in retinal pigment cells in an effective amount to prevent, reduce, or alleviate one or more symptoms of age-related macular degeneration or diabetic retinopathy.

In some embodiments, a compound disclosed herein is administered to a subject in an effective amount to treat or prevent one or more condition, signs, or symptom associated with sickle cell disease, a beta-thalassemia, or a related disorder as provided below.

Beta-thalassemia can include symptoms such as anemia, fatigue and weakness, pale skin, or jaundice (yellowing of the skin), protruding abdomen with enlarged spleen and liver, dark urine, abnormal facial bones and poor growth, and poor appetite.

In subjects with sickle cell disease, or a related disorder, physiological changes in RBCs can result in a disease with the following signs: (1) hemolytic anemia; (2) vaso-occlusive crisis; and (3) multiple organ damage from microinfarcts, including heart, skeleton, spleen, and central nervous system.

In sickle cell disease, red blood cell survival is around 10-20 days. Approximately one third of the hemolysis occurs intravascularly, releasing free hemoglobin (plasma free hemoglobin [PFH]) and arginase into plasma. PFH has been associated with endothelial injury including scavenging nitric oxide (NO), proinflammatory stress, and coagulopathy, resulting in vasomotor instability and proliferative vasculopathy.

Vaso-occlusive crisis occurs when the circulation of blood vessels is obstructed by sickled red blood cells, causing ischemic injuries. The most common complaint is of pain, and recurrent episodes may cause irreversible organ damage. One of the most severe forms is the acute chest syndrome which occurs as a result of infarction of the lung parenchyma.

Pain can affect any body part. It often involves the abdomen, bones, joints, and soft tissue, and it may present as dactylitis (bilateral painful and swollen hands and/or feet in children), acute joint necrosis or avascular necrosis, or acute abdomen. The liver also may infarct and progress to failure with time. Papillary necrosis is a common renal manifestation of vaso-occlusion, leading to isosthenuria (i.e., inability to concentrate urine).

Severe deep pain can be present in the extremities, involving long bones. Abdominal pain can be severe, resembling acute abdomen; it may result from referred pain from other sites or intra-abdominal solid organ or soft tissue infarction.

Bone pain and abdominal pain may be present. The face also may be involved. Pain may be accompanied by fever, malaise, and leukocytosis. Skeletal manifestations include, but are not limited to, infarction of bone and bone marrow, compensatory bone marrow hyperplasia, secondary osteomyelitis, secondary growth defects, intravascular thrombosis, osteonecrosis (avascular necrosis/aseptic necrosis), degenerative bone and joint destruction, osteolysis (in acute infarction), Articular disintegration, periosteal reaction (unusual in the adult), Dystrophic medullary calcification, bone-within-bone appearance, decreased density of the skull, decreased thickness of outer table of skull due to widening of diploe, hair on-end striations of the calvaria, osteoporosis sometimes leading to biconcave vertebrae, coarsening of trabeculae in long and flat bones, and pathologic fractures, bone shortening (premature epiphyseal fusion), epiphyseal deformity with cupped metaphysis, peg-in-hole defect of distal femur, and decreased height of vertebrae (short stature and kyphoscoliosis).

Renal manifestations include, but are not limited to, various functional abnormalities such as hematuria, proximal tubule dysfunction, impaired potassium excretion, and hyperkalemia; and gross anatomic alterations, for example, hypertrophied kidneys, with a characteristic smooth, capsular surface.

Splenic manifestations include, but are not limited to, enlargement, including rapid and/or painful enlargement known as splenic sequestration crisis, infarction, low pH and low oxygen tension in the sinusoids and splenic cords, functional impairment, fibrosis and shrinking of the spleen, immune deficiency, and increased risk of sepsis.

In certain embodiments, this disclosure relates to using a compound disclosed herein for treating subjects with sickle cell disease, beta thalassemia, or variants or related diseases or conditions thereof.

In certain embodiments, this disclosure relates to using a compound disclosed herein for reducing one or more symptoms of sickle cell disease, beta thalassemia, or variants or related diseases or conditions thereof.

In certain embodiments, this disclosure relates to using a compound disclosed herein for treating a sickle cell-related disorder.

In certain embodiments, this disclosure relates to using a compound disclosed herein for treating sickle cell-related retinopathy.

In certain embodiments, the method further comprises administering hydroxyurea or another therapeutic agent to the subject. In certain embodiments, the subject is unresponsive to treatment with hydroxyurea or another therapeutic agent alone. In certain embodiments, the compound disclosed herein is administered in combination hydroxyurea or another therapeutic agent when the subject is unresponsive or does not respond well to treatment with hydroxyurea or another therapeutic agent alone.

In certain embodiments, this disclosure relates to using a compound disclosed herein for treating sickle cell disease resulting in fewer, or less severe side effects, greater efficacy, greater response rate, or combinations thereof compared to existing therapies such as hydroxyurea or another therapeutic agent.

In certain embodiments, the compound disclosed herein is used in combination or alternation with another therapeutic agent to treat sickle cell disease or complications. Representative additional therapeutic agents include, but are not limited to, L-glutamine, 1-(butyryloxy)ethyl-5-amino-4-oxopentanoate (AN-233), crizanlizumab, voxelotor, i.e., chemical 2-hydroxy-6-((2-(1-isopropyl-1H-pyrazol-5-yl) pyridin-3-yl) methoxy)benzaldehyde, name fumaric esters, pain relieving drugs, hydroxyurea, and combinations thereof.

In certain embodiments, administering a compound disclosed herein may be done before, during or after red blood cell (RBC) transfusion or a hematopoietic stem cell transplantation (HSCT).

In certain embodiments, any of the methods reported herein may be used in combination with providing a subject with fluids (e.g., pH balanced isotonic salt solution), oxygen, analgesics, or combinations thereof.

In certain embodiments, the disclosed methods include administering to the subject an active agent, for example, vitamin supplements, nutritional supplements, anti-anxiety medication, anti-depression medication, anti-coagulants, clotting factors, anti-inflammatoiresteroids such as corticosteroids, analgesic, etc. In certain embodiments, active agents may include folic acid, penicillin, or another antibiotic, preferably a quinolone or macrolide, antivirals, anti-malarial prophylactics, and analgesics to control pain.

Methods of selecting a subject with a mutation in a beta-globin gene for treatment are also disclosed. The methods typically include genotyping the beta-globin gene and expression control sequence thereof in DNA isolated from a biological sample obtained from the subject; determining if the beta-globin gene or expression control sequence includes a mutation; selecting the subject for treatment if the beta-globin gene or expression control sequence includes a mutation; and treating the subject with an effective amount of a compound disclosed herein.

### Pharmaceutical Compositions

In certain embodiments, this disclosure relates to pharmaceutical compositions comprising a compound as disclosed herein or salt thereof and a pharmaceutically acceptable excipient. In certain embodiments, the pharmaceutical composition is in the form of a tablet, pill, capsule, gel, gel capsule, or cream. In certain embodiments, the pharmaceutical composition is in the form of a sterilized pH buffered aqueous salt solution or a saline phosphate buffer between a pH of 5 to 9, optionally comprising a saccharide or polysaccharide.

In certain embodiments, the pharmaceutical composition is in solid form surrounded by an enteric coating. In certain embodiments, the enteric coatings comprises a component such as methyl acrylate-methacrylic acid copolymers, cellulose acetate phthalate (CAP), cellulose acetate succinate, hypromellose (hydroxypropyl methylcellulose), hypromellose phthalate (hydroxypropyl methyl cellulose phthalate), hypromellose acetate succinate (hydroxypropyl methyl cellulose acetate succinate), diethyl phthalate, polyvinyl acetate phthalate (PVAP), methyl methacrylate-methacrylic acid copolymers, or combinations thereof.

In certain embodiments, the pharmaceutically acceptable excipient is selected from lactose, sucrose, mannitol, triethyl citrate, dextrose, cellulose, microcrystalline cellulose, methyl cellulose, ethyl cellulose, hydroxyl propyl cellulose, hydroxypropyl methylcellulose, carboxymethylcellulose, croscarmellose sodium, polyvinyl N-pyrrolidone (crospovidone), ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, the pharmaceutical composition is in the form of a saline, citrate buffer or phosphate buffer, optionally comprising a saccharide or polysaccharide.

In certain embodiments, this disclosure relates to pharmaceutical compositions comprising a compound disclosed herein or pharmaceutically acceptable salt, and a pharmaceutically acceptable vehicle, adjuvant, or carrier.

In certain embodiments, the pharmaceutical composition comprises a compound disclosed herein and a propellant. In certain embodiments, an aerosolizing propellant is compressed air, ethanol, nitrogen, carbon dioxide, nitrous oxide, hydrofluoroalkanes or combinations thereof.

In certain embodiments, the disclosure contemplates a pressurized or unpressurized container comprising a compound herein. In certain embodiments, the container is a manual pump spray, inhaler, meter-dosed inhaler, dry powder inhaler, nebulizer, vibrating mesh nebulizer, jet nebulizer, or ultrasonic wave nebulizer.

In certain embodiments, the pharmaceutical compositions may additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants.

The pharmaceutical compositions containing compounds reported herein may further comprise one or more additional therapeutic agent(s).

In certain embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levofloxacin, including aerosolized formulations thereof, and combinations of two antibiotics, e.g., fosfomycin and tobramycin.

In another embodiment, the pharmaceutical compositions can be administered to a patient once daily or about every twenty-four hours. Alternatively, the pharmaceutical compositions can be administered to a patient twice daily. Alternatively, the pharmaceutical composition of the invention can be administered about every twelve hours. These pharmaceutical compositions are administered as oral formulations containing about 25 mg, 50 mg, 100 mg, 125 mg, 150 mg, 200 mg, 250 mg, 300 mg, or 400 mg of a compound disclosed herein. In this aspect, the pharmaceutical compositions further comprise a filler; a disintegrant; a surfactant; a binder; or a lubricant, or combinations thereof.

### Identification of Anti-Sickling Agents by Screening a Compound Library Via an Imaging-Based High Throughput Assay

An imaging assay utilizing automated microscopy and customized algorithms was used to detect sickled or normal (round shaped) RBCs. The assay is capable of discovering anti-sickling agents with physiologically relevant oxygen affinity. The assay uses native mouse or human RBCs, enabling one to identify a broader spectrum of agents and is not restricted to compounds that require direct interaction (e.g., with purified hemoglobin). Thus, in contrast to hemoglobin binding assays, this approach allows for the identification of additional classes of anti-sickling agents that modulate pathways affecting RBC sickling. Compounds resulting from the experiments are also useful for investigating SCD pathophysiology.

The imaging-based protocol that can distinguish sickled vs normal (round) phenotype of intact RBCs originating from genetically engineered mice expressing human hemoglobin genes (the HbSS Townes knock-in sickle mice), or 2) SCD patients. The imaging was conducted using a BioTek Cytation™ cell imaging multimode reader and software to detect sickled or normal round-shaped phenotype of RBCs with negative control (vehicle treatment) and a positive control, 5-hydroxymethylfurfural (5-HMF).

### Cytotoxicity Assay

To screen out anti-sickling agents that cause cell death, LDH-glo Cytotoxicity Assay Kit (Promega) was used to measure lactate dehydrogenase activity in the media which is released from the cytoplasm of cultured cells due to breach in the plasma membrane. Some of the compounds in the pilot assay show significant toxicity to HEK293 cells after 24 h incubation. Others exhibited negligible cytotoxic effects.

### General Procedure-1 for Synthesis of Maleimides

To a 25 ml round bottom flask equipped with a magnetic stirbar and under an inert atmosphere of nitrogen/argon was added maleic anhydride 1 (2.0 equiv). Glacial Acetic Acid was then added to obtain a 0.5 M mixture that was stirred at rt until complete dissolution of the maleic anhydride. Using a syringe, the corresponding aniline 2 (1.0 equiv) was added dropwise. The reaction flask was fitted with a Findenser™ and the reaction mixture was set to stir at reflux overnight (about 16 hours). After indication by TLC (5% Ethyl Acetate/DCM) that the aniline has been consumed, the reaction mixture was allowed to cool down as it continued to stir. The slightly warm reaction mixture was then poured unto ice. The solid crude precipitate was then transferred unto Buchner filtration funnel and washed with cold DI water. The obtained solid was redissolved in hot ethanol and recrystallized overnight in the freezer. The crystalline product is then washed with hexanes and dried under high vacuum to obtain the desired HDYB maleimide.

### General Procedure-2 for Synthesis of Oxindolones

To a 25 ml round bottom flask equipped with a magnetic stir bar and under an inert atmosphere of nitrogen/argon was added Isatin (1H-indole-2,3-dione) 3 (1.0 equiv), the corresponding benzyl bromide 4 (1.0 equiv) and K2CO3 (3.0 equiv) were heated at reflux in acetonitrile for 1-2 hours. After completion of the reaction as indicated by TLC (2% MeOH/Hexanes), the reaction mixture was allowed to cool down to room temperature and the solvent was removed by reduced pressure before being extracted with ethyl acetate. The combined organic extract was dried over MgSO₄ and concentrated to obtain the clean desired HDYB product with no further purification.

TABLE 1
shows data on Anti-sickling activity of maleimides derivatives

| | | | Anti-sickling activity (EC50; μM) | |
|---|---|---|---|---|
| | 2D structure | Chemical name | ss Mouse RBC | Human RBC |
| HDYB-1 | 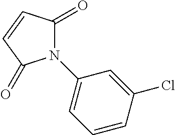 [SMILES: O=C1C=CC(=O)N1c1cccc(Cl)c1] | | 5 | 6.3 |
| HDYB-2 | 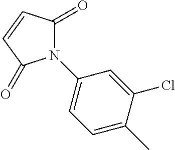 | | 15 | 19 |
| HDYB-3 | 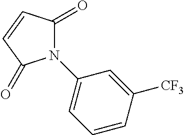 [SMILES: O=C1C=CC(=O)N1c1cccc(C(F)(F)F)c1] | | 12 | 12 |
| HDYB-4 | 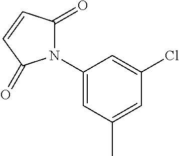 | | 33 | 18 |
| HDYB-5 | 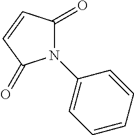 [SMILES: O=C1C=CC(=O)N1c1ccccc1] | | 21 | 17 |
| HDYB-6 | 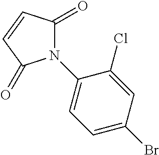 [SMILES: O=C1C=CC(=O)N1c1ccc(Br)cc1Cl] | | 20 | 12 |
| HDYB-7 | 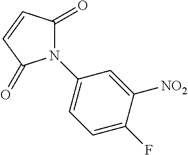 [SMILES: O=C1C=CC(=O)N1c1ccc(F)c([N+](=O)[O-])c1] | | 23 | 45 |
| HDYB-8 | 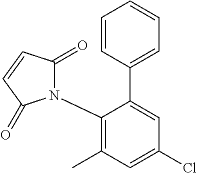 [SMILES: Cc1cc(Cl)cc(-c2ccccc2)c1N1C(=O)C=CC1=O] | | <100 | <100 |
| HDYB-9 | 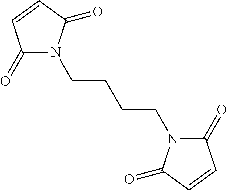 [SMILES: O=C1C=CC(=O)N1CCCCN1C(=O)C=CC1=O] | | 29 | 78 |
| HDYB-10 | 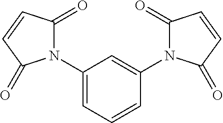 [SMILES: O=C1C=CC(=O)N1c1cccc(N2C(=O)C=CC2=O)c1] | | 78 | 57 |
| HDYB-11 | 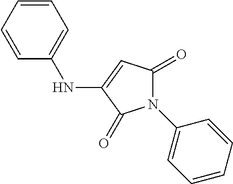 [SMILES: O=C1C=C(Nc2ccccc2)C(=O)N1c1ccccc1] | | <100 | <100 |
| HDYB-12 | 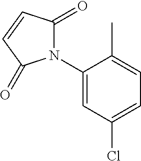 | | 16 | 12 |
| HDYB-13 | 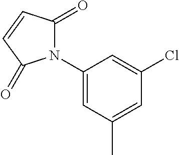 | | 22 | 22 |
| HDYB-14 | 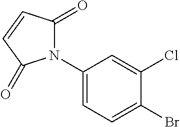 [SMILES: O=C1C=CC(=O)N1c1ccc(Br)c(Cl)c1] | | 27 | 23 |
| HDYB-15 | 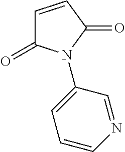 [SMILES: O=C1C=CC(=O)N1c1cccnc1] | | <100 | <100 |
| HDYB-16 | 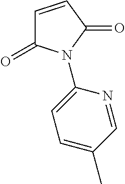 | | <100 | <100 |
| HDYB-17 | 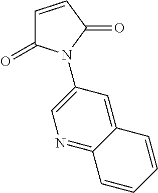 [SMILES: O=C1C=CC(=O)N1c1cnc2ccccc2c1] | | <100 | <100 |
| HDYB-18 | 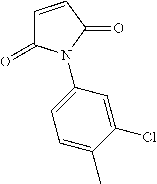 | | 52 | 52 |
| HDYB-19 | 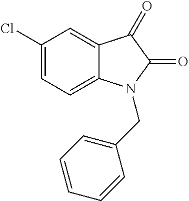 [SMILES: O=C1C(=O)N(Cc2ccccc2)c2ccc(Cl)cc21] | | 27 | 25 |
| HDYB-20 | 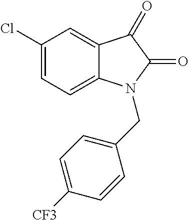 [SMILES: O=C1C(=O)N(Cc2ccc(C(F)(F)F)cc2)c2ccc(Cl)cc21] | | 22 | 26 |
| HDYB-21 | 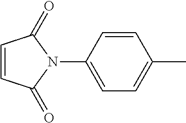 | | 37 | 32 |
| HDYB-22 | 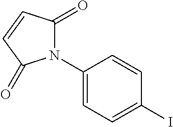 [SMILES: O=C1C=CC(=O)N1c1ccc(I)cc1] | | 13 | 41 |
| HDYB-23 | 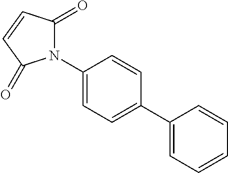 [SMILES: O=C1C=CC(=O)N1c1ccc(-c2ccccc2)cc1] | | 68 | 52 |
| HDYB-24 | 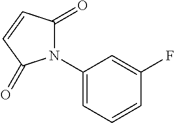 [SMILES: O=C1C=CC(=O)N1c1cccc(F)c1] | | 11 | 14 |
| HDYB-25 | 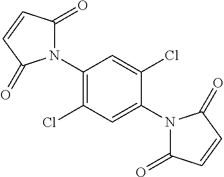 [SMILES: O=C1C=CC(=O)N1c1cc(Cl)c(N2C(=O)C=CC2=O)cc1Cl] | | 10 | 12 |
| HDYB-26 | 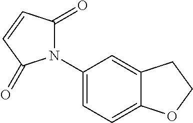 [SMILES: O=C1C=CC(=O)N1c1ccc2c(c1)CCO2] | | 9 | 13 |
| HDYB-27 | 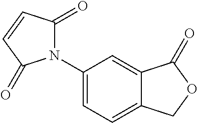 [SMILES: O=C1OCc2ccc(N3C(=O)C=CC3=O)cc21] | | 10 | 11 |

### Animal Studies of Compounds for Managing of Sickle Cell Disease

High throughput drug screening to identify molecules that prevent sickling of diseased RBCs. The resulting hits will be verified in dose response and a series of secondary assays. Lead compounds will be selected based on physiologically acceptable changes of hemoglobin oxygen affinity and RBC membrane deformability. Lead scaffolds will be synthesized and evaluated for anti-sickling potency, hemoglobin oxygen affinity, and improved RBC membrane deformability to identify agents best suited for animal safety and efficacy studies. Townes HbSS mice expressing human globin genes will be used to test short term lethality, efficacy of anti-sickling activity, RBC oxygen affinity, and RBC half-life in vivo.

Compounds with therapeutic potential for sickle cell disease (SCD) will be intraperitoneally injected into B6/129 or C57BL/6 mice at different doses. Body weight and clinical signs of the mice will be monitored to find the short-term (3-day) maximum tolerated dose (MTD) of each test compound and pharmacokinetics (PK) will be explored. The 3-day MTD will be used as a basis in B6/129 or C57BL/6 mice to determine a more chronic MTD (e.g., 3 or 4 week administrations) for each test compound useful for conducting efficacy studies in B6/129 or C57BL/6 mice with sickle cell disease.

TABLE 2
Sickle Cell Disease Compounds EC50s

| | EC50 for anti-sickling activity (uM, |
|---|---|
| Compound_ID | normalized to positive control of 5 mM 5-HMF) |
| SCAS2_01 | 1.1 |
| SCAS2_02 | 25.4 |
| SCAS2_05 | 2.25 |
| SCAS2_11 | 15.2 |
| SCAS2_61 | 3.12 |

Anti-sickling activity (%, normalized
to positive control of 5 mM 5-HMF)

| Compound_ID | 100 uM | 33 uM | 10 uM |
|---|---|---|---|
| SCAS2-A2 | 213.95 | 25.26 | 21.45 |
| SCAS2-A13 | 35.39 | 90.158 | 71.04 |
| SCAS2-A14 | 131.87 | 78.71 | 24.26 |

What is claimed is:

1. A compound having the following formula 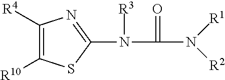
or salt thereof wherein
R¹ is hydrogen, alkyl, halogenated alkyl, formyl, alkanoyl, benzyl, benzoyl, carbamoyl, sulfamoyl, carbocyclyl, aryl, or heterocyclyl, wherein R¹ is optionally substituted with one or more, the same or different, R¹⁰;
R² is hydrogen, alkyl, halogenated alkyl, formyl, alkanoyl, benzyl, benzoyl, carbamoyl, sulfamoyl, carbocyclyl, aryl, or heterocyclyl, wherein R² is optionally substituted with one or more, the same or different, R¹⁰; or
R¹ and R² and the attached nitrogen come together to form a carbocyclyl, aryl, or heterocyclyl optionally substituted, with one or more, the same or different, R¹⁰;
R³ is hydrogen, alkyl, halogenated alkyl, formyl, alkanoyl, benzyl, benzoyl, carbamoyl, sulfamoyl, carbocyclyl, aryl, or heterocyclyl;
R⁴ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁴ is optionally substituted with one or more, the same or different, R¹⁰;
R⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁵ is optionally substituted with one or more, the same or different, R¹⁰;
or R⁴ and R⁵ and the attached atoms come together to form a cyclic or heterocyclic ring optionally substituted with one or more, the same or different, R¹⁰;
R¹⁰ is alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, cyclopropylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

2. The compound of claim 1 having the following formula 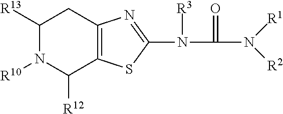
or salt thereof wherein
R¹ is hydrogen, alkyl, halogenated alkyl, formyl, alkanoyl, benzyl, benzoyl, carbamoyl, sulfamoyl, carbocyclyl, aryl, or heterocyclyl, wherein R¹ is optionally substituted with one or more, the same or different, R¹⁰;
R² is hydrogen, alkyl, halogenated alkyl, formyl, alkanoyl, benzyl, benzoyl, carbamoyl, sulfamoyl, carbocyclyl, aryl, or heterocyclyl, wherein R² is optionally substituted with one or more, the same or different, R¹⁰; or
R¹ and R² and the attached nitrogen come together to form a carbocyclyl, aryl, or heterocyclyl optionally substituted, with one or more, the same or different, R¹⁰;
R¹⁰ is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R¹⁰ is optionally substituted with one or more, the same or different, R¹¹;
R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, cyclopropylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;
R¹² is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl; and
R¹³ is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl.

3. The compound of claim 1 having the following formula 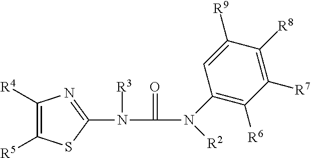
or salt thereof wherein
R² is hydrogen, alkyl, halogenated alkyl, formyl, alkanoyl, benzyl, benzoyl, carbamoyl, sulfamoyl, carbocyclyl, aryl, or heterocyclyl, wherein R² is optionally substituted with one or more, the same or different, R¹⁰; or
R³ is hydrogen, alkyl, halogenated alkyl, formyl, alkanoyl, benzyl, benzoyl, carbamoyl, sulfamoyl, carbocyclyl, aryl, or heterocyclyl;
R⁴ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁴ is optionally substituted with one or more, the same or different, R¹⁰;
R⁵ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁵ is optionally substituted with one or more, the same or different, R¹⁰;
R⁶ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁶ is optionally substituted with one or more, the same or different, R¹⁰;
R⁷ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁷ is optionally substituted with one or more, the same or different, R¹⁰;
R⁸ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁸ is optionally substituted with one or more, the same or different, R¹⁰;
R⁹ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁹ is optionally substituted with one or more, the same or different, R¹⁰;
R¹⁰ is alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, cyclopropylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

4. The compound of claim 1 having the following formula 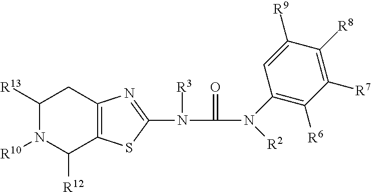
or salt thereof wherein
R² is hydrogen, alkyl, halogenated alkyl, formyl, alkanoyl, benzyl, benzoyl, carbamoyl, sulfamoyl, carbocyclyl, aryl, or heterocyclyl, wherein R² is optionally substituted with one or more, the same or different, R¹⁰; or
R³ is hydrogen, alkyl, halogenated alkyl, formyl, alkanoyl, benzyl, benzoyl, carbamoyl, sulfamoyl, carbocyclyl, aryl, or heterocyclyl;
R⁶ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁶ is optionally substituted with one or more, the same or different, R¹⁰,
R⁷ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁷ is optionally substituted with one or more, the same or different, R¹⁰;
R⁸ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁸ is optionally substituted with one or more, the same or different, R¹⁰,
R⁹ is hydrogen, alkyl, halogenated alkyl, halogen, hydroxy, alkoxy, alkylthio, amino, alkylamino, (alkyl)₂amino, cyano, formyl, alkanoyl, benzyl, benzoyl, carboxy, carbamoyl, sulfamoyl, carbocyclyl, aryl, or heterocyclyl, wherein R⁹ is optionally substituted with one or more, the same or different, R¹⁰;
R¹⁰ is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, cyclopropylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;
R¹² is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl; and
R¹³ is hydrogen, alkyl, halogen, nitro, cyano, hydroxy, amino, mercapto, formyl, carboxy, carbamoyl, sulfamoyl, alkoxy, alkylthio, alkylamino, (alkyl)₂amino, alkylsulfinyl, alkylsulfonyl, carbocyclylsulfonyl, arylsulfonyl, heterocyclylsulfonyl, alkanoyl, benzyl, benzoyl, carbocyclyl, aryl, or heterocyclyl.

5. The compound of claim 4, which is 2-(3-(5-chloro-2-methoxyphenyl)ureido)-N,N-dimethyl-6,7-dihydrothiazolo[5,4-c]pyridine-5 (4H)-sulfonamide (SCAS2_01) or salt thereof.

6. The compound of claim 4, which is 1-(5-chloro-2-methoxyphenyl)-3-(5-(cyclopropylsulfonyl)-4,5,6,7-tetrahydrothiazolo[5,4-c]pyridin-2-yl)urea (SCAS2_05) or salt thereof.

7. A compound 6-(piperidin-1-ylsulfonyl)benzo[d]thiazol-2-amine (SCAS-02) or salt thereof.

8. A pharmaceutical composition comprising a compound of claim 7 and a pharmaceutically acceptable excipient.

9. A method of treating or preventing sickle cell disease or related condition comprising administering an effective amount of a compound of claim 7 to a subject in need thereof.

10-20. (canceled)
